(19) United States
(12) Patent Application Publication
(10) Pub. No.: US 2006/0167420 A1
(43) Pub. Date: Jul. 27, 2006

(54) Device for protection of a needle of a medical device

(76) Inventor: Vincent Saldell, Akersberga (SE)

Correspondence Address: JACOBSON HOLMAN PLLC, 400 SEVENTH STREET N.W., SUITE 600, WASHINGTON, DC, 20004, US

(21) Appl. No.: 10/563,825

(22) PCT Filed: Jul. 9, 2004
(86) PCT No.: PCT/SE04/01124

§ 371 Date: Jan. 6, 2006

(30) Foreign Application Priority Data
Jul. 9, 2003 (SE) 0302036.9

(51) Int. Cl. A61M 5/32

(52) U.S. Cl. 604/192

(57) ABSTRACT

Device for protection of a needle (6) for a medical device (7), that can enclose the needle (6) in a first position (13) and leave the needle (6) free in a second position (14). The device includes a first part (1) and a second part (2) which are pivotally connected by a joint device (12). The first part (1) at a distance from said joint device (12) includes holding means (5) for retaining the medical device (7) pivotally about an axis that is essentially parallel with a joint axis of said joint device (12), so that the two parts (1,2) can be brought together to the first position (13) for enclosing the needle and can be separated from each other to the second position (14) where a retained needle (6) is pivotable to its free position (14). The invention also concerns a medical device.

### FIELD OF THE INVENTION

The present invention relates to a device according to the preamble of claim 1.

### PRIOR ART

Risk of blood infection in connection with vein needles, injection syringes and iv cannulae is every day life for medical staff world wide. Needlesticks with contaminated needle may imply transfer of a number of serious deceases, with focus on HIV, hepatitis B and C. In the USA San Francisco Journal in 1998 published statistics that death as a cause of an occupational injury being four times more common among medical and health care staff than among policemen.

There is to this day no vaccine for HIV, and HIV can not be cured. Development to AIDS can only be restrained with so called anti retroviral medicines. Hepatitis B is very infectious, but today there are well functioning vaccines. With adult patients the decease in 5% of the cases leads to a chronic hepatitis B, which has an aggressive form that often leads to cirrhosis of the liver or liver cancer. As things are at present, there is no vaccine for hepatitis C and moreover, according to the Swedish Smittskyddsinstitutet, at least 50% of the patients develop a chronic hepatitis C, for which one also can see a quite large part of cirrhosis of the liver and liver cancer.

Today treatment of both chronic hepatitis B and C is tried out with antiviral drugs, often in combination with interferon. For certain treatment combinations of chronic hepatitis C an improved or completely normalized liver function is observed with almost half of the treated cases.

In all the world 40 million people are today carrying HIV and 170 million people hepatitis. In Africa, liver cancer and AIDS are the leading causes of death with people between 30 and 45 years of age.

Probability for infection after a needlestick incident with contaminated needle is dependent on several factors, among others what pathogen is involved, susceptibility of the affected person, the degree of needlestick injury, amount of blood/pathogen and if possible prophylaxis after the needlestick is used.

In an extensive global study of risks for infections by HIV-virus at needlestick incidents it turned out that the transfer frequency on average was 0.3%. However, studies of needlestick incidents with high risk reveal a considerably higher degree of transfer. Incidents with high risk are for example needlesticks where medical staff will come in contact with a greater amount of blood, if the needlestick was deep or if the procedure was to place the needle in the vein or artery of the patient.

The risk of infection for needlestick incidents concerning hepatitis B is 6-30% after one single needlestick. However individuals that are vaccinated do not run any risk. Follow up treatment is also possible with more than 90% probability of avoiding infection.

For hepatitis C the probability of infection is up to 7%. Unfortunately, there is no suitable prophylaxis to use after the needlestick and, as mentioned earlier, no vaccine.

In the USA alone it is estimated that between 600.000 and 800.000 percutaneous needlestick and cut incidents occur annually among medical staff. [NIOSH/U.S. Department of Health and Human Sevices: “Alert Preventing Needlestick Injuries in Health Care Settings”].

Even though the risks for infection of hepatitis B can be diminished with medication, the best protection against HIV and hepatitis C is to prevent the needlestick incident.

As an example of state of the art of a device for protection against needlesticks, EP 0 819 441 A1 could be mentioned, which discloses a syringe with cover. A cover is covering the needle of a syringe, and the cover can be unfolded from and folded back to the needle. In covering position the cover prevents the needle from causing injury and in uncovered position the cover can be fastened, so that it can not move and interfere at the moment of injection. Moreover, by means of the known device it is possible to identify an already used syringe, even when the cover has been returned to the folded, protecting position.

This construction has several deficiencies. At the very moment of the needlestick this device does not protect the user. For example, at emergency wards in hospitals it often occurs that the staff must treat individuals that are under the influence of drugs or by any other reason unruly. The user is completely unprotected in this hazardous situation. Studies also reveal that approximately as many needlestick incidents occur at the actual needlestick moment as after. Moreover, this construction is of the active type. That is, after the treatment of the patient the protection must be activated to be effective.

The Purpose of the Invention and its Most Important Characteristics

It is a purpose of the present invention to provide a solution of or reduction of the prior art problems and thus, by simple means, obtain a protection for a user against needlesticks that protects during the complete treatment occasion; before, during and after the needlestick on the patient. Moreover, the protection should be of passive nature; it should be effective all the time, without needing manual activation. It is also desirable that it can offer protection for different types of medical devices provided with needles.

According to the invention this is achieved by a device of the type mentioned in preamble by the distinctive features that are disclosed in the characterising portion of claim 1.

By needles (hollow bore needle) is in this document understood all oblong objects, with an inner canal, which can penetrate organic tissue.

By way of the invention a passive protection is accomplished that protects during the complete treatment occasion by means of the needle, for example a syringe, a vein needle or an intra venous (iv) cannula, being enclosed by two protective parts, a first and a second, according to the invention, that forms surfaces towards the user which the needle can not penetrate or get beside.

The user may hold the second part with his one hand in a holder means and the medical device with the needle with the other hand. Alternatively, the holder means may include a fixing means that is fastened to the patient. The user may then hold the medical device with the needle with his one hand, whereby thus the other hand of the user is free.

In a first position the two parts enclose the needle and this is then completely protected by these parts. The user may now bring the two parts from each other in that these being pivotally connected by a joint device. The needle, which is pivotally suspended by a means at the first of these parts is now released to a second position and is ready to be used. The second part is provided with an end part which can be placed onto the skin of the patient, to give the device increased stability.

The point of the needle may essentially describe a circular arc when it is moving in its pivotal suspension in the first part. Both parts are covering the complete circumference of this circular arc, except for that part of the circular arc that faces the patient. The user is thus protected while the point of the needle can be brought to the skin of the patient.

When the needle has been used, the device with the needle is folded again, in the opposite way that it was unfolded.

One or both parts may have a recess in which the needle fits when the device according to the invention is in the first folded protective position. This arrangement replaces the conventional needle cap.

It is possible to design the holding means to be separate from the first part and fasten these together with a second joint device. As a suggestion, the holding means are fitted with a recess or a bore through which the needle can be introduced and fastened, for example by snapping or screwing on. Nevertheless it should be mentioned that the needle also can be produced integrally with the holding means, for example if the device according to the invention should be delivered together with the needle.

The device according to the invention will be differently designed depending upon which type of needle it should be used with. The needles differ on the one hand from each other physically in size, which causes that the device must be adapted accordingly. Moreover, different types of needles are used in different ways. For example, the angle of inclination to the skin of the patient is varying greatly for an injection syringe and an iv cannula. To control this angle of inclination and to give the device according to the invention more stability, this can be provided with means which limits the space of both parts and the needle around their joint axes.

The surfaces of both parts can be fitted with recesses or grooves, which by means of the capillary force may absorb blood. In that way the risk that the user comes in contact with the blood of the patient is further reduced. It is also possible to consider to use, as an alternative and complement for grooves, an absorbing material, such as a super absorbent.

The end part of the second part, which is applied to the skin of the patient can be fitted with a contact surface of a material with high friction, so that the device according to the invention does not slip.

As an alternative, an adhesive material, such as tape, can be used to fix the second part to the skin of the patient. As a fixing means a string, a strap, a ring or a fastening flap can also be used.

If needed the second part could be fitted with a particular penetration protection. It is suitably designed in metal, tough plastic or other material that is difficult to penetrate for the needle.

The two parts may need to be fastened to each other in the first position: for transport, or, after using the needle, to, for example, tightly seal the parts around the needle. This can be achieved in that the two needles are being fitted with snap lock elements, which engages each other and holds said parts together. Other forms of means that keeps the two parts together are also possible, such as Velcro fastening or adhesive material, for example.

The needle in connection with the invention can thus be an injection syringe, a vein needle or an iv cannula. However, it is of course possible to use the invention with all possible equipment that is sufficiently small and that includes a needle, with the purpose to protect a user of such equipment against needlestick incidents.

The two joint devices can be composed of flexible material bridges or joints with pivot pins. The holding means, for the hand of the user, can, as an example, be a ring.

Certainly it is imaginable to combine the invention with medical equipment including a needle, so that they form a unit together.

Additional distinctive features and advantages of the invention are evident from the following exemplifying description.

### BRIEF DESCRIPTION OF THE DRAWINGS

Embodiments that exemplify the invention will now be described by reference to the following drawings, on which:

FIG. 1 illustrate a first embodiment of the invention,

FIGS. 2a and b illustrate two positions that can be occupied by a device according to the invention,

FIGS. 3a and b illustrate a snap lock of the invention by which a medical device may be snapped tight,

FIG. 4 illustrates diagrammatically angles that arise in connection with the invention,

FIGS. 5a and b illustrate a device according to the invention which includes recesses in the form of grooves for the absorption of blood,

FIGS. 6a and b illustrate two different variants of an end part of the invention,

FIGS. 7a and b illustrate snap elements by which the parts of the device can be locked together,

FIGS. 8a, b and c show a structure of the device in which an iv cannula can be used,

FIG. 9 illustrates a second embodiment of the device according to the invention,

FIGS. 10a and 10b, respectively, illustrate locking means by which the needle may be locked inside a part of the invention, and

FIG. 11 illustrates a security device according to the invention.

### DESCRIPTION OF EMBODIMENTS

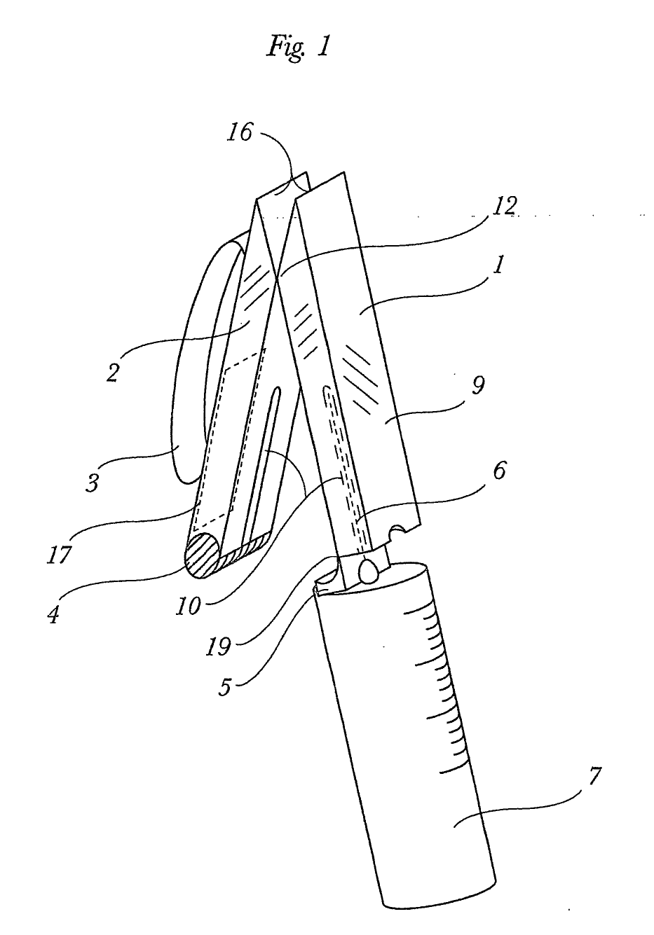

FIG. 1 illustrates a device for protection of a needle of a medical device. The device includes two parts 1, 2, which are connected to each other by a joint device 12. A medical device 7 is pivotally suspended at the first part 1, at a distance from the joint device 12.

In a first embodiment of the invention, at the second part 2, a holder means 3 is arranged, intended for a hand of a user. In this case the holder means is a handle or a ring for the introduction of at least one finger of the user.

In a second embodiment of the invention, instead the device is fastened at the patient by a fixing means 23, by which the second part 2 is fixed at the patient. In this way it becomes possible for the user to use the device according to the invention by one hand.

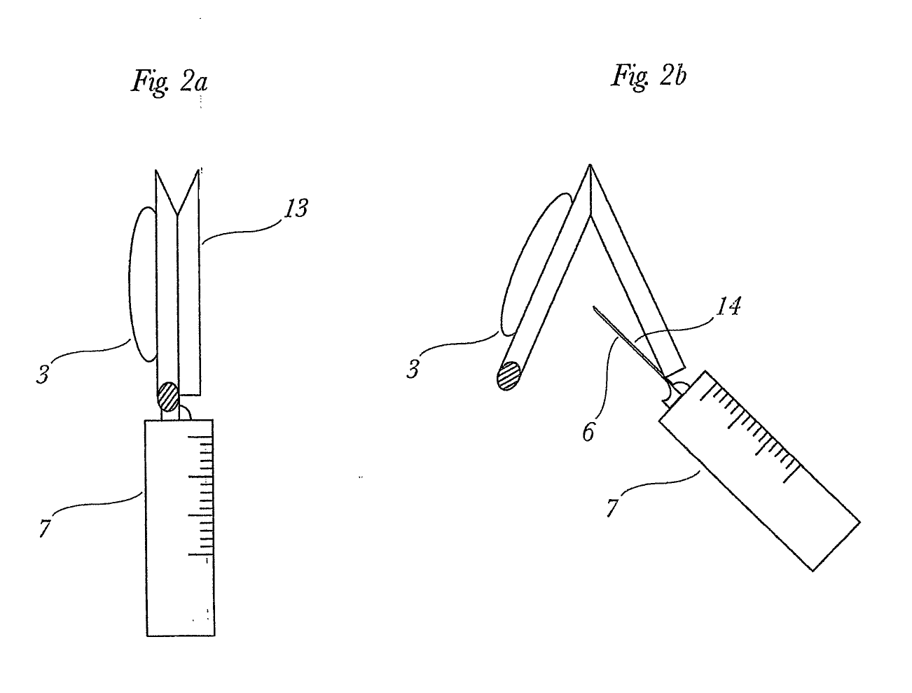

The device may at least be brought to two different positions. At the first position 13 (FIG. 2), the two parts enclose the needle 6, whereby this is protected. In the second position 14 (FIG. 2b), the needle 6 is left free and thus enabled to be used.

The handling of the first embodiment is done according to the following. Before the use of the device, this is folded in the first position 13 (FIG. 2a), whereby the needle is protected from getting in contact with, for instance, the user. He grasps the holder means 3 with his one hand and the medical device 7 with his other. The user now brings the two parts 1, 2 away from each other, turnably around the joint device 12. As the two parts 1, 2 are leaving each other; the needle 6 is left free and can move around its pivotable suspension. The end 4 of the second part 2 may now be placed against the skin of the patient, for increased stability, and the needle 6 is ready to be used.

The two joint axes, which arise by the joint device 12 and the pivotal suspension of the medical device 7 are essentially parallel. By way of that the construction of the device is made in other respects sufficiently rigid, a system with only two degrees of freedom will be achieved for the medical device 7. Thus, this can only describe a plane that is perpendicular to the joint axes. Since the parts 1, 2 of the device enclose the needle all the time in this plane and in those directions that are facing the user and his hands; he is protected all the time by these parts 1, 2 against needlestick incidents.

The only direction in which the needle 6 is not enclosed by the two parts 1, 2 is the one that is facing the patient. In this manner it is possible for the needle 6 to be brought to the skin of the patient.

The user is now moving the needle to such a position that it has the right angle of incident against the skin of the patient, for the desired type of needlestick. The whole time the user is holding his one hand at the second part 2 and his other hand at the medical device 7. The needlestick is given, the needle then being removed and in an inverse manner brought back to the protective first position 13.

The pivotable connection of the medical device can be accomplished in many ways. A first variant is to use a joint. Another variant (not shown) the needle is fitted, in right angle to this, with an axis which can be suspended in therefore intended bores in the first part 1. The pivotal action thereby arises, by that the axis can be turned in the bores. A third variant (not shown either) could be to fit the first part 1 with a narrow slit, in which the needle 6 fits and can travel. By making the slit narrow and with small extension, the desired pivotal action in one plane arises.

Moreover, on the second part there is a holder means 3, in which the user can hold the device according to the invention. The holder means 3 may, for example, be in the form of a loop, a handle, a ring, gripping surface or any other type of means for the hand and/or the fingers of the user. Its function is to keep the hand and the fingers of the user away from the area to which the point of the needle may reach.

One or both parts 1, 2 may be fitted with a complementary recess 10, in which the needle fits in the closed position 13. In this way, both parts 1, 2 may completely enclose the needle 6.

FIGS. 2a and b illustrate the first position 13 and the second position 14 respectively, of the device. In the position 13 the needle is enclosed by the device and completely protected from contact. In position 14 the needle is free to move and may thus be applied at a patient.

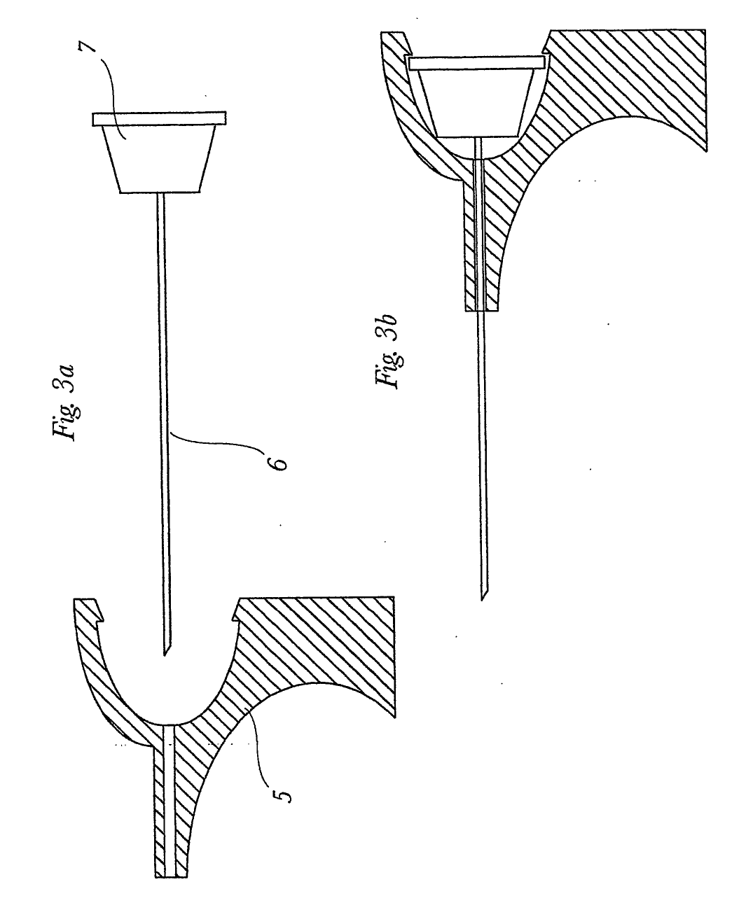

FIGS. 3a and b illustrate a holding means 5 and a needle 6. In this case the holding means 5 is arranged separately from part 1 and is fixed to this by way of a (second) joint device 19 (FIG. 1). The fastening means 5 may also be integrated with the first part 1. It is advantageous to provide the holding means 5 with a recess or bore in which the needle fits. In the figures, a snap lock mechanism is designed at the holding means 5, for snap locking the medical device 7. By the snap lock mechanism the device may be manufactured in a non-disposable type: after use the needle may be snapped loose, the device be disinfected and a new needle may be snapped on. Of course, it is possible to use the snap lock mechanism with disposable types of the device. Other types of fastening of the medical device 7 to the holding means 5 are of course, imaginable, for example by screw tightening, gluing, and melting. In the case of screw tightening, a standard thread at a medical device 7 may be used to, in a simple and rational way, fasten this medical device 7 at the holding means 5.

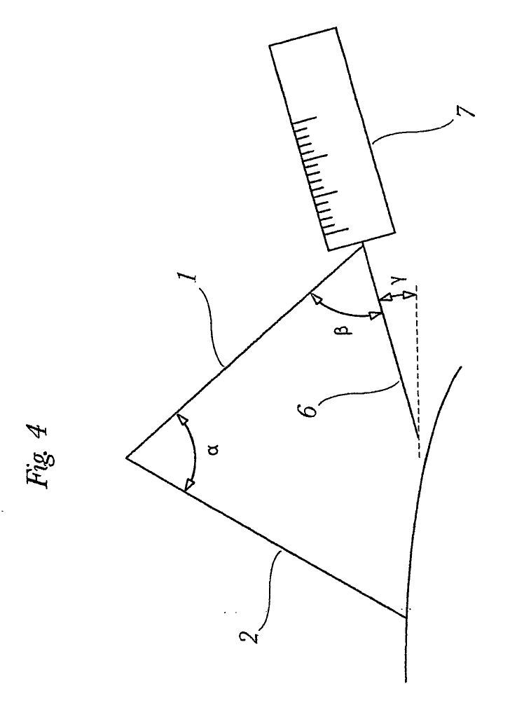

FIG. 4 illustrates diagrammatically the device with two angles α and β, which arises between the different parts. Depending on the size and type of the medical device one wants to be able to achieve different angles α between the parts 1 and 2 and β between the first part 1 and the needle 6 at the needlestick occasion on the patient. For example, with an injection syringe there is often a need for the angle γ of incident of the needle to be around 45-80° against the skin of the patient. With a vein needle or an iv cannula one strives for a more flat angle γ of incidence, often about 10-30°. To achieve these angles, α and β are varied accordingly. In typical cases a may be within the interval of 30-60° and β within the interval of 50-80°.

To provide the device with increased stability, increase the precision and to prevent incorrect extremes, the device may be fitted with means to limit one or both angles α, β. As an example of such means the bevellings 16 on the device in FIG. 1 could be mentioned. They function in such a way that the two parts 1, 2 may move away from each other until the two bevellings 16 meet, whereby a natural stop occurs. A similar arrangement, with surfaces that meet and limit the mobility may be arranged at the fastening of the medical device 7 at the first part 1.

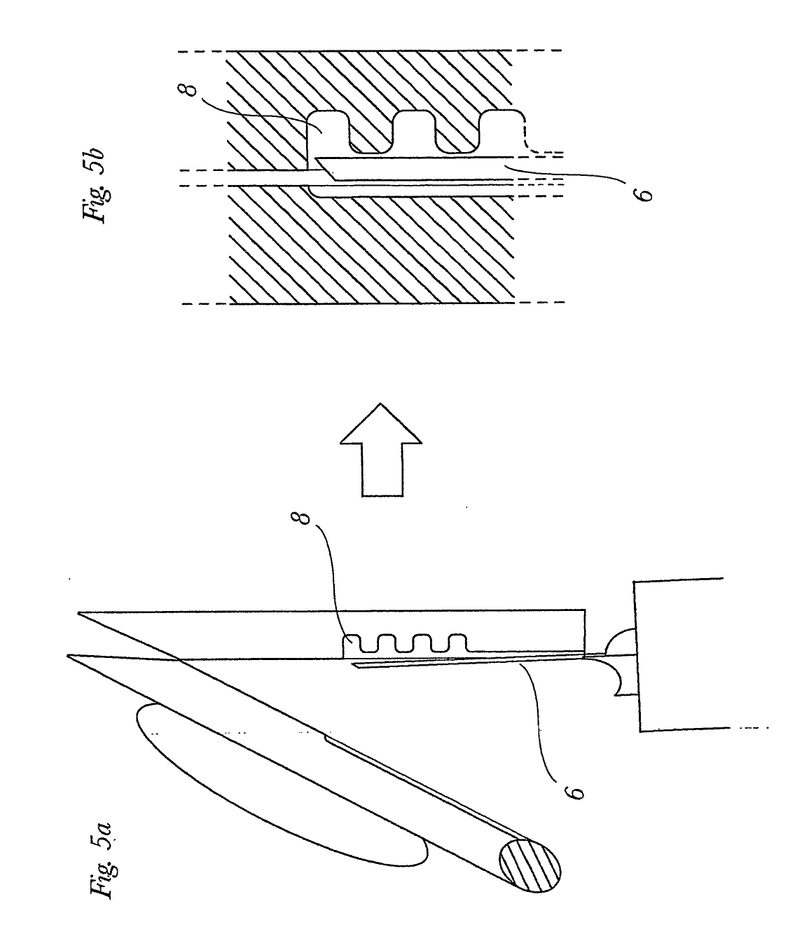

FIGS. 5a and b illustrate an embodiment of the invention where sections of the parts 1, 2 have been fitting with recesses in the form of grooves 8, which by the capillary force may absorb blood. If, for example, the needle after the needlestick on the patient is filled with blood, these grooves may catch such blood, to avoid the blood to be spilled out from the device. As an alternative or complement for grooves, an absorbing material, such as for example a super absorbent may be used, which also acts as an absorbent.

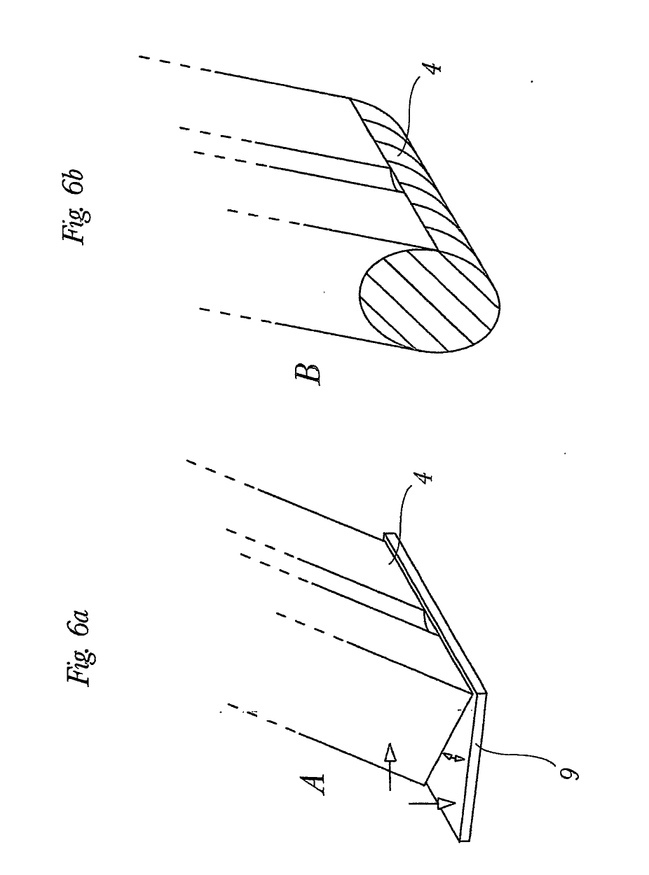

FIGS. 6a and b illustrate two different variants A, B of the end part 4 of the device. At the first embodiment A, the end part is fitted with an adhesive material at the contact surface with the patient, for instance in the form of an end plate. In this way a good anchoring of the device on the patient is ensured. The adhesive material is preferably some sort of medical tape.

With the other variant B, a contact surface of a high friction material is used instead. This material may, for example, be rubber. Likewise this has the function to ensure a good anchoring of the device to the patient. Of course, with this embodiment, an adhesive material could also be used.

Possibly, the device could be fitted with a penetration protection 17 (FIG. 1), in the case the material as such in the second part 2 does not for sure withstand a penetration attempt by the needle. The penetration protection may be made of metal, for instance, a tough plastic or other material that is difficult to penetrate for the needle. It may be joined together with the second part 2 by embedding, gluing, melting or other suitable method.

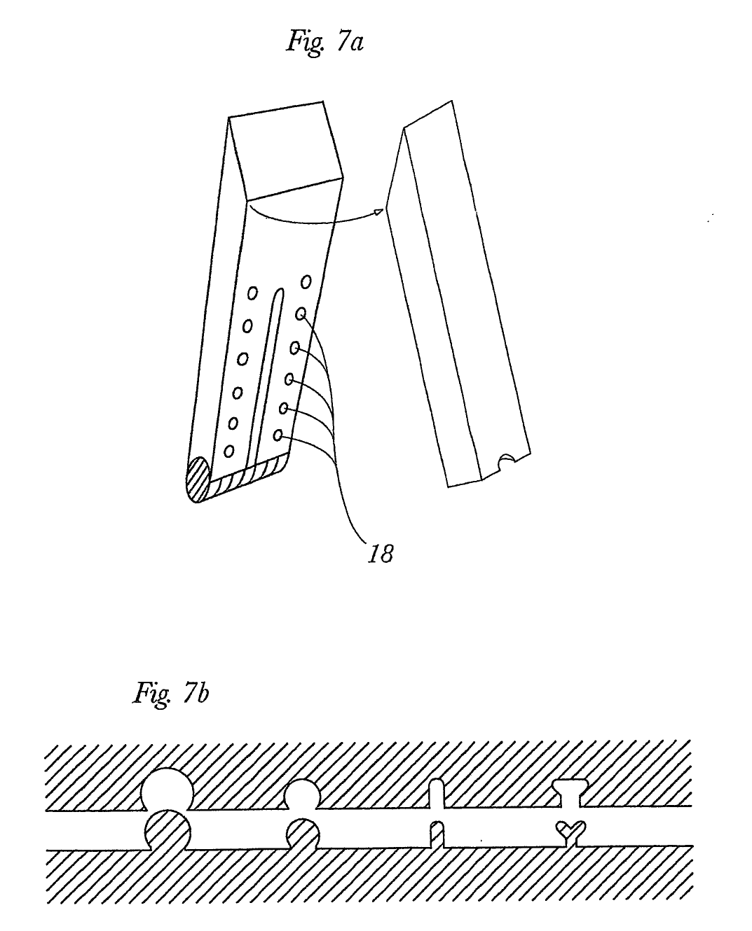

FIGS. 7a and b illustrate examples of mutual snap locking of the two parts 1, 2. To fasten the two parts 1, 2 against each other in the protective position 13; these can be fitted with some sort of snap lock. In the Figure, snap lock elements 18 are illustrated arranged on either part 1, 2 which elements 18 engage each other. These may be designed in different ways, of which some are shown in the Figure. Other types of snap locking are also possible, for instance by strips (not shown) situated at the side of the parts 1, 2, which can engage with each other. Instead of snap lock means, it is possible to use other means for keeping the parts together, for instance Velcro tape or an adhesive means. It is of course possible to keep the two parts 1, 2 together by means of other measures, such as an elastic string.

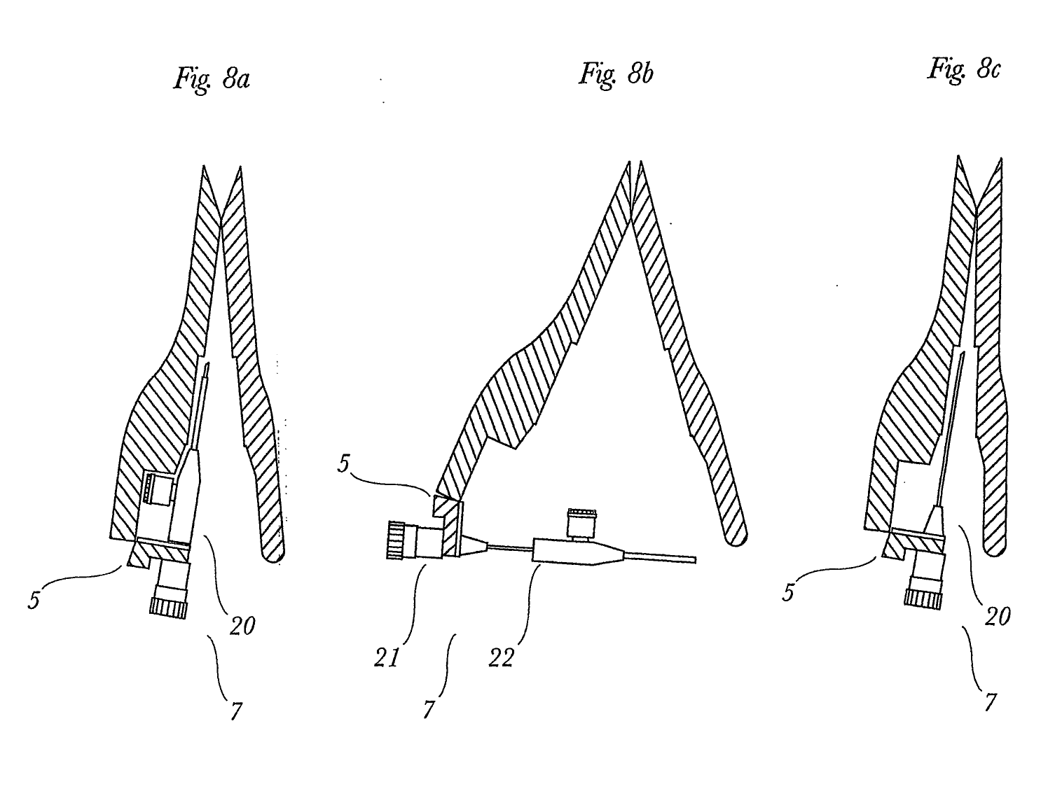

FIGS. 8a, b and c illustrate the device in relation to an iv cannula 20. The iv cannula 20 is constructed of two different parts, stylet 21 and catheter 22. As the catheter 22, after the needle placement, shall remain with the patient the iv cannula must be fastened at the holding means 5 in the stylet 21. This may result in that the part of the medical device 7 that must be enclosed by the device will become longer than in other cases. This makes that the device correspondingly must be made longer. In c the device is shown with removed catheter, after the application of this catheter.

If an injection syringe or a vein needle is used as a medical device including a needle, the construction of the device is adapted accordingly, to match these.

The joint devices 12, 19 may be made of flexible material bridges of synthetic material, for instance. These material bridges may be made integrally with the device according to the invention or as a separate means, which is fastened in a suitable way at both parts 1, 2. The thickness of the material bridge may be chosen according to the desired flexibility. With a broader material bridge a virtual joint axis may arise, which is that axis around which both pivotal parts are turning. Another example of the design of the joint devices 12, 19 is a pivot pin device. The joint devices 12, 19 may also be designed with a plurality of axes, which in that case forms a virtual axis around which both pivotal parts are turning.

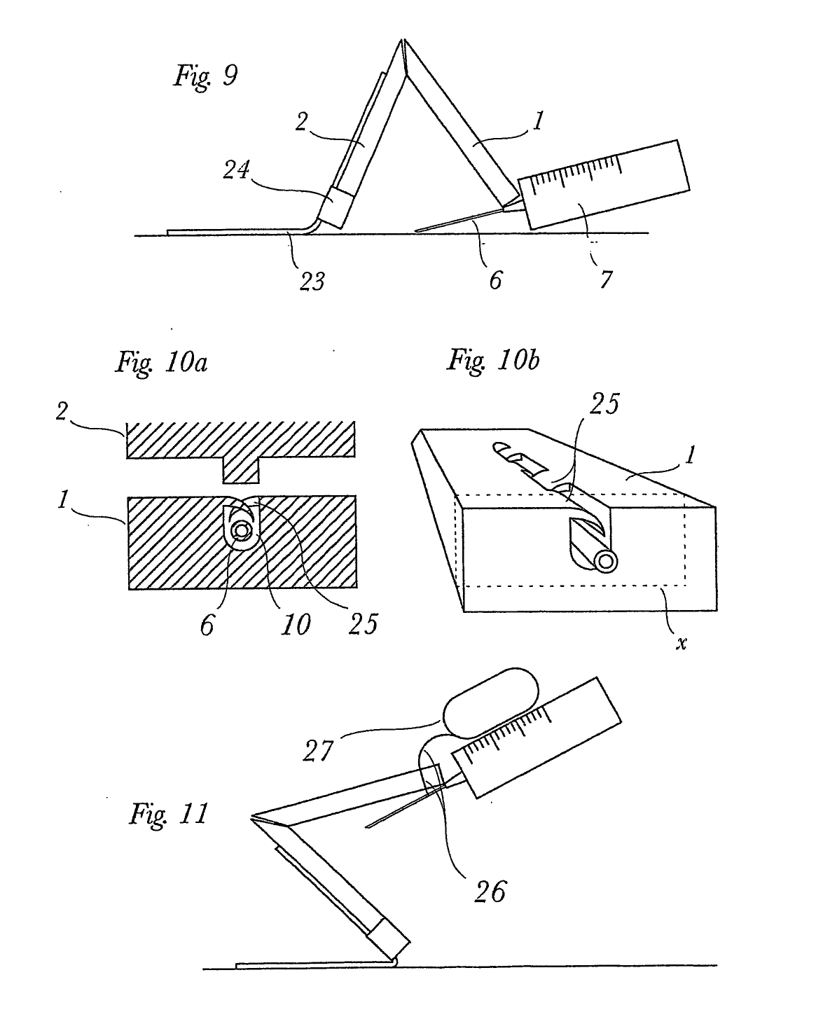

The fixing means 23 attachable to the patient in the second embodiment (see FIG. 9) may be made of, for instance, tape, a string, a strap, a ring, or a fastening flap. The fundamental idea is that the second part and therewith the whole device according to the invention is fastened in a steady manner to the patient, so that the user does not have to use both hands to handle said device. After use of the device according to the invention the fixing means 23 may be detached from the patient in a suitable manner, depending upon what type of fixing means is concerned. It is also possible that at least a part of the means 23 is left on the patient, for example a tape can be designed in such a way that a part of the tape remains on the device according to the invention and a part remains on the patient, by means of a part of the fixing means 23 is detachably fastened at the second part 2. If such a part is provided with plaster fabric and remains on the patient, it may serve the purpose of a plaster. A fastening flap is, for example, a thin flap in a suitable material that is being applied to the patient and then is fixed on him with an external means, for instance, a strap. A stabilising means 24 may, if required, be used to retain the fixing means 23 at the second part 2 during the use of the device according to the invention. The stabilising means 24 may be made in synthetic material, such as plastic, or every other suitable material. If the fixing means 23 shall be removed from the second part 2, for instance after use, the stabilising means 24 may be detached by, for example, cutting this means or by undoing snaps on this means.

As an alternative to the above, a separate holder or fixing means may be used, that at the occasion of use is fastened both at the patient and at the part 2 in a suitable manner, for example by taping up.

The above mentioned different alternatives in fastening the device according to the invention on the patient may also be combined with the above mentioned adhesive end part A. For example, the means 23 may be combined with this end part A. Both means may be separate or made integrally with each other.

There is nothing that prevents use of a holder means 3 in connection with the fixing means 23 of the second embodiment. Even though the use of the device according to the second embodiment may take place by means of only one hand, a holder means 3 may form a suitable gripping surface for the user at the handling of the device according to the invention.

FIG. 10 illustrates a cross-section of FIG. 10b in the plane that has been marked with X in FIG. 10b. Both figures illustrates a variant where a complementary recess 10 may be provided with means 25 that after use of the needle 6, locks it in a protected position inside the complementary recess 10. This means may be made of, for example, flaps 25 that has been placed along each side of the length of the recess 10, covering the recess 10. Such flaps may be constructed in a material that is non-rigid in one direction and rigid in another. In that way the flaps bend and let the needle enter the recess, but in the other direction the flaps are rigid and blocks the needle. Hereby the needle 6 is fixed inside the recess 10. Other constructions with the same fundamental idea may of course be envisaged.

As an additional security measure, it is proposed a means 26 that at an unexpected movement of the patient automatically pulls in the needle in the protective recess 10. See FIG. 11. Such a means 26 may, for instance, be a thread, a cord or strip that is fastened at or in the neighbourhood of the needle, in front of that joint axis that arise at the second joint device 19, in the direction of the needle. This means then extends along the first part 1, from its inside to its outside, for example through a bore in this part 1. On the outside of the first part 1, the means 26 forms a loop 27, or other suitable form that can be fixed at the user, for instance at the hand holding the needle. In the case of a loop 27 this can be made in that the user simply runs his hand through the loop 27. If the user by an accident looses grip of the medical device 7, the needle will, as soon as the hand of the user is removed from the device 7, automatically be pulled into the recess 10 of the first part 1. This is done by means 26, described in this paragraph, pulling the needle 6 around said joint axis into the recess 10.

The invention also includes a medical unit, which includes a medical device 7 with a needle. For example both may be delivered as a pre-packaged sterile unit.

The invention is not limited by the above examples, but can be varied within the scope of protection defined in the claims. As an example, one of both parts 1, 2 of the device may be made transparent, which gives the user a better view when using the device. As a variant of view improving measures it is possible to equip the first part 1 of the device with one or more perforations.

The form and the size of one or both parts 1, 2 of the device may be varied in many ways, as long as these protect the user from the needle. For example, the second part 2 may be made relatively large, to provide a good protection.

Due to which type of needle that is used the fastening in the invention is adapted accordingly.

The thickness of material of the device depends on the material chosen and the desired rigidity of the invention. All materials that are sufficiently rigid and are suitable for medical use may be used, for instance plastic or metal.

1. A device for protection of a needle for a medical device that includes a first part and a second part, which are movably connected, the first part including a holding means for retaining the medical device, so that the two parts can be brought together to a first position for enclosing the needle and can be separated from each other to a second position where a retained needle is in a free position, characterised in that
the first part is pivotally connected about an axis to the holding means,
the second part is pivotally connected about an axis to the first part at a distance from the holding means,
said axes are essentially parallel and sufficiently rigid to form a system in which the medical device with the needle only can describe a plane that is perpendicular to the joint axes, whereby the two parts are capable of protecting the user from the tip of the needle in directions facing the user before, during and after the needlestick on the patient, and
the second part includes a holder means for fixing said second part.

2. The device according to claim 1, characterized in that at least one of the parts in closed position has a recess complementary to the needle.

3. The device according to claim 1, characterised by that the holding means is separate from the first part and fastened to the first part with a (second) joint device.

4. The device according to claim 1, characterised in that the holding means has a recess for receiving and retaining said needle.

5. The device according to claim 1, characterised in that the holding means is fitted with a mechanism for retaining the medical device, selected from the group consisting of: snap lock device, screw device.

6. The device according to claim 1, characterised by means to limit an angle (α) between the first part and the second part in the separated position and/or an angle (β) between the first part and the needle in pivoted position.

7. The device according to claim 6, characterised in that the angle (α) is about 30°-60°.

8. The device according to claim 6, characterised in that the angle (β) is about 50°-80°.

9. The device according to claim 1, characterised in that one or both parts includes at least one recess, designed to, by the capillary force, absorb blood and/or an absorbing material.

10. The device according to claim 1, characterised in that the second part has an end part for application on a patient, provided with a contact surface of a material with large friction or an adhesive material, such as tape.

11. The device according to claim 1, characterised in that the second part is reinforced with a penetration protection, of metal or of tough plastic, for example.

12. The device according to claim 1, characterised in that the first and second part of the device has a locking mechanism in the form of cooperating snap lock elements, Velcro tape or adhesive material, to, in the first position, secure the parts to each other.

13. The device according to claim 1, characterised in that medical device is a vein needle, an injection syringe or an iv cannula with stylet.

14. The device according to claim 1, characterised in that the joint device is a device selected from the group consisting of: flexible material bridge, pivot pin device.

15. The device according to claim 3, characterised in that the second joint device is a device selected from the group consisting of: flexible material bridge, pivot pin device.

16. The device according to claim 1, characterised in that the holder means is a handle for a user.

17. The device according to claim 1, characterised in that said holder means includes a fixing means that is attachable to the body of a patient.

18. The device according to claim 1, characterised in that the fixing means is any means selected from the group consisting of: tape, string, strap, ring, fastening flap.

19. The device according to claim 18, characterised in that at least a section of the fixing means is detachably fastened to the second part.

20. The medical unit comprising a medical device with a needle, characterised in that it includes a device according to claim 1.
